(19) United States
(12) Patent Application Publication
(10) Pub. No.: US 2003/0129634 A1
(43) Pub. Date: Jul. 10, 2003

(54) General screening method for ligand-protein interactions

(76) Inventors: David R.H. Evans, Guilford, CT (US); Jan Fredik Simons, New Haven, CT (US); Kendra Swirsding, Middlefield, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/289,738

(22) Filed: Nov. 7, 2002

Related U.S. Application Data
(60) Provisional application No. 60/344,861, filed on Nov. 7, 2001.
(60) Provisional application No. 60/335,606, filed on Nov. 15, 2001.

(51) Int. Cl. C12Q 1/68; C12Q 1/04

(52) U.S. Cl. 435/6; 435/34

(57) ABSTRACT

The invention provides a rapid method of performing yeast hybrid assays by detecting the activation of at least three reporter genes using detection assays specific for each reporter gene that are performed in one container.

### RELATED APPLICATIONS

The instant application claims priority to Provisional Application U.S. S. No. 60/344,861, filed Nov. 7, 2001 and U.S. S. No. 60/335,606, filed Nov. 15, 2001, both of which are incorporated herein by reference in their entirety.

### FIELD OF THE INVENTION

The present invention relates to generalized methods for performing yeast hybrid assays.

### BACKGROUND OF THE INVENTION

Knowledge of the protein-protein interactions occurring within a cell provides insight into gene function and nominates proteins for pharmaceutical modulation. The yeast two-hybrid (Y2H) system (Fields and Song, Nature 340: 245-6, 1989) has been used to chart the complete interactome of a phage, a bacterium and a yeast (Bartlet, et al., Nat. Genet. 12: 72-77, 1996; Rain et al., Nature 409: 211-15, 2001; Uetz, et al, Nature 403: 623-27, 2000; Ito, et al., Proc. Natl. Acad. Sci. USA 98: 4569-74, 2001). The Y2H system relies on the interaction of two fusion proteins to bring about the transcriptional activation of a reporter gene such as E. coli derived β-galactosidase (Lac Z). Commonly, gene X (the bait) is fused to the DNA binding domain (BD) of the yeast transcription factor Gal4, while gene Y (the prey) is fused to the Gal4 activation domain (AD). If the two hybrid proteins interact in vivo (in a Y2H host cell) their binding unites the BD and AD of Gal4, thereby reconstituting a functional transcription factor. The protein-protein interaction is, in turn, detected by a reporter gene expressed under the control of a Gal4-inducible promoter (generally GAL_{UAS}-lacZ, encoding the bacterial β-galactosidase and/or a GAL_{UAS}-driven yeast prototrophic gene such URA3). Yeast cells harboring an activated reporter gene can be differentiated from other cells and the cDNA encoding for the interacting polypeptides can be easily isolated and sequenced.

The application of Y2H assay has been adapted to screening of peptide combinatorial libraries and protein interactions (Meijia et al. Nucl. Acids Res. 23, 1152 (1995)). However, this assay is unsuited for screening small molecule-protein interactions because it relies solely on genetically encoded fusion proteins. Furthermore, for organisms with large genomes, novel techniques that enhance the veracity and throughput of the Y2H system are required to uncover protein-protein interactions comprehensively.

The most commonly used reporters include genes required for prototrophic cell growth in the absence of a particular amino acid or base, and the bacterial lacZ gene encoding β-galactosidase (reviewed in Serebriiskii, et al., BioTechniques 30: 634-55, 2001). In addition, some strains of Saccharomyces cerevisiae contain the MEL1 gene (Aho, et al., Anal. Biochem. 253: 270-72, 1997), that is expressed under the control of the Gal4 transcription factor and codes for a secreted α-galactosidase enzyme (Lilijestrom, Nuc. Acids Res. 13: 7257-68, 1985; Buckbolz and Adams, Mol. Gen. Genet. 182: 77-81, 1981). Thus, MEL1 provides an endogenous reporter gene compatible with the Gal4-based Y2H system (Aho, et al., Anal. Biochem. 253: 270-72, 1997). Specific assays for the separate analysis of α- and β-galactosidase activity have been described (Buckholz and Adams, Mol. Gen. Genet. 182: 77-81, 1981; Lazo, et al., Eur. J. Biochem. 77: 375-82, 1977; Ryan, et al., Mol. Cell. Biol. 18: 1774-82, 1998; Melcher, et al., Gene 247: 53-61, 2000) and lacZ has been used as a reporter in large-scale, automated screens (Buckholz, et al., J. Molec. Microbiol. Biotech. 1: 135-40, 1999).

The related yeast three-hybrid (Y3H) system was developed to detect the interaction between proteins and small molecule ligands (Licitra, et al., Proc. Natl. Acad. Sci. USA 93: 12817-21, 1996). In this system the Gal4_{BD}-bait and Gal4_{AD}-prey fusion-proteins do not interact in vivo, but instead they bind different small molecule ligands. A hybrid small molecule, consisting of the ligand for the bait protein chemically linked to the ligand for the prey, can form a bridge between bait and prey thereby re-constituting the Gal4 transcription factor, which in turn leads to reporter-gene activation.

Through the synthesis of hybrid small-molecule libraries the Y3H system has the potential to identify the ligand(s) of a given protein receptor. Conversely, through expression of prey cDNA libraries in a suitable yeast host strain, the Y3H system can be used to identify the protein receptor of a given ligand (provided that the ligand can be linked to a second, known ligand that binds a suitable bait). Thus, the Y3H system presents opportunities for pharmaceutical development through its capacity to identify novel drugs and drug targets.

Detection of Y3H reporter gene-activation is generally performed on selective agar medium containing a chromogenic substrate for β-galactosidase (Licitra, et al., Proc. Natl. Acad. Sci. USA 93: 12817-21, 1996; Lin, H, et al. J. Am. Chem. Soc. 122: 4247-4248, 2000). However, this approach has several drawbacks including the long incubation period required for cell growth and color development (3 days to 1 week) and the difficulty in quantifying reporter gene readout. Furthermore, the preparation of agar medium containing the required drug concentrations is arduous and time consuming, while the solid nature of agar medium prohibits the use of liquid handling robots for automated screening.

### SUMMARY OF THE INVENTION

The invention disclosed herein provides a rapid method and kit for identifying the targets of biologically active small molecules so as to identify new drugs that are capable of specific therapeutic effects as well as to identify novel small molecules including agonists and antagonists that may bind selected targets.

In one aspect, the invention is directed to a method of performing a yeast hybrid assay. The method includes detecting the activation of at least three reporter genes using detection assays specific for each reporter gene, wherein the detection assays are all performed in the same container. The yeast hybrid assay can be either a yeast two hybrid assay or a yeast three hybrid assay. The reporter genes can be URA3, MEL1, and/or lacZ. The detection assay used to detect the reporter genes assays can operate through measuring the activity of a protein product of the reporter gene. For example, the protein products can be orotidine-5′-phosphate decarboxylase, α-galactosidase and/or β-galactosidase. The detection assay used to detect the reporter genes may also detect the presence of the protein product of the reporter gene.

According to the invention, the assays are performed on a surface other than a solid or a gel. In one embodiment, the assays can not be performed on a surface, wherein that surface is a solid or a gel. The assays can be performed in a container in a liquid medium. The container can be a well or a microtiter plate. The microtiter plate can have 24, 96, 384, 1536, or more wells.

In another aspect, the invention is directed to a method of performing a yeast two hybrid assay, by detecting the interaction of a prey fusion protein and a bait fusion protein by the activation of at least three reporter genes each detected by specific detection assay, wherein at least three detection assays are performed in one container. For example, the reporter genes can be URA3, MEL1, and/or lacZ. The detection assays used to detect the reporter genes can measure the activity of a protein product of the reporter gene. This protein product can be orotidine-5′-phosphate decarboxylase, α-galactosidase and/or β-galactosidase. The detection assay may also be used to detect the presence of the protein product of the reporter gene.

The assays are performed on a surface other than a solid or a gel, such as a liquid medium. In one embodiment, the assays can not be performed on a surface, wherein that surface is a solid or a gel. The container can be a well or a microtiter plate. The microtiter plate can have 24, 96, 384, 1536, or more wells.

In another embodiment, the invention is directed to a method of performing a yeast three hybrid assay, by detecting the interaction of a prey fusion protein, a ligand, and a bait fusion protein by the activation of at least three reporter genes each detected by specific detection assay, wherein at least three detection assays are performed in one container. For example, the reporter genes can be URA3, MEL1, and/or lacZ. The detection assays used to detect the reporter genes can measure the activity of a protein product of the reporter gene. This protein product can be orotidine-5′-phosphate decarboxylase, α-galactosidase and/or β-galactosidase. The detection assay may also be used to detect the presence of the protein product of the reporter gene.

The assays can be performed under conditions other than on a surface such as a solid or a gel; for example, the assays can be performed in a liquid medium. A container for a liquid medium can be a well or a microtiter plate. The microtiter plate can have 24, 96, 384, 1536, or more wells.

### BRIEF DESCRIPTION OF THE DRAWINGS

FIG. 1 is a diagrammatic representation of the yeast two hybrid assay showing the interaction between a protein X DNA binding domain (Gal4 or LexA) fusion protein and a protein Y activation domain fusion protein, expressed by cDNA, which triggers the expression of the reporter gene (His 3, LacZ, Ura3) subsequent to the interaction of the transcriptional activator modules with the Gal 4/LexA upstream activating sequences.

FIG. 2 is a diagrammatic representation of the components of the three-hybrid assay showing a known target protein (X) DNA binding domain (Gal4 or LexA) fusion protein, and protein Y activation domain fusion protein, expressed by cDNA, and the hybrid ligand A-B that interacts with the two fusion proteins X (A interacts irreversibly with X) and Y (reversible interaction) resulting in the activation of the reporter genes ((His 3, LacZ, Ura3) subsequent to the interaction of the transcriptional activator modules with the Gal 4/LexA upstream activating sequences.

FIG. 3A is a graph showing the α-galactosidase activity of select diploid colonies measured by sequential galactosidase detection of protein-protein interactions using the Gal4-based Y2H system. FIG. 3B is a graph of the β-galactosidase activity of select diploid colonies measured by sequential galactosidase detection of protein-protein interactions using the Gal4-based Y2H system. Incubation time for the α- and β-galactosidase assay was 90 min and 120 min, respectively. Data for each strain are the average (±S.D.) of assays performed in triplicate and corrected for background signals produced in the absence of a Y2H interaction (strain CGY-4D).

FIG. 4A is a graph comparing α-galactosidase activity of select diploid colonies generated by either interaction mating (open bars) or by growing diploid cells directly without mating (closed bars) as measured by sequential galactosidase detection of protein-protein interactions using the Gal4-based Y2H system. FIG. 4B is a graph comparing β-galactosidase activity of select diploid colonies generated by interaction mating (open bars) or by growing diploid cells directly without mating (closed bars)as measured by sequential galactosidase detection of protein-protein interactions using the Gal4-based Y2H system. In each case the number of diploid cells assayed for α- and β-galactosidase activity was approximately 5.5×10⁵ (per well). Incubation time for the α- and β-galactosidase assay was 180 min and 60 min, respectively. Data for each strain are the average (±S.D.) of assays performed in triplicate and corrected for background signals produced in the absence of a Y2H interaction (strain CGY-4D).

FIG. 5A is a graph showing interaction of rGR and FKBP12 mediated by dex-FK506 in cells inoculated into medium lacking uracil as determined by the drug-dependent trans-activation of GAL_{UAS}URA3 reporter gene. FIG. 5B is a graph showing interaction of rGR and FKBP12 mediated by dex-FK506 in cells inoculated into medium lacking uracil as determined by the drug-dependent trans-activation of GAL_{UAS}-lacZ reporter gene. The data are for strain CGY-201-202D and were corrected for background levels of reporter gene activity produced by the negative control strain, CGY-201-GHD, which contained rGR but not FKBP12. For (FIG. 5B) an aliquot (50 μl) of each cell culture was transferred from a 96-well plate to a 384-well plate, an equal volume of CPRG assay buffer was added and the ΔA₅₇₈/A₆₆₀ was measured following color-development at room temperature for 5 h. Similar results were obtained if an equal volume of CPRG assay buffer was added to cultures directly in the 96-well growth plate.

### DETAILED DESCRIPTION

According to the invention, the term “screening assay”, as used herein, is as a process for selecting or eliminating items by means of at least one distinctive criteria. The screening assay is intended to be distinct from any assay of biological function or effect. The items in this method are small molecules, and the selection is based on capability of binding a target molecule (sometimes called a receptor). A feature of the screening assay is the ability to rapidly examine the binding of large numbers of different small molecules for selected target molecules and, conversely, to examine the binding of selected molecules for a large number of target molecules. The positive interaction between small molecules and a target results in a chemical signal that is quantitatively and/or qualitatively different from a signal if any produced in the negative control.

“The sample containing an environment”, as used herein, is defined as a sample containing a complex biochemical mixture such as is found within a eukaryotic or prokaryotic cell or alternatively may be formed from a cell lysate maintained in a synthetic boundary such as a membrane or a reaction vessel.

“A cellular component” is defined herein as including a nucleic acid, a polysaccharide, a lipid, or a protein or any combination of these.

A “reporter gene” or a “reporter” is defined herein as a marker for detecting the formation of a hybrid complex. The reporter is not intended in itself to have a therapeutic effect in the environment within which it is located in the assay.

An “interactor colony” is a haploid strain of yeast that has either a “bait” protein/DNA binding domain fusion protein encoding plasmid or a “prey” protein/transactivation domain protein containing plasmid. These two types of interactor colony members are mated to produce diploid yeast cells that have both types of plasmids within them.

The invention is generally drawn to methods of performing a yeast hybrid assay by measuring the activity of the MEL1 and lacZ reporter genes, along with the cell density associated with the activity of the URA3 gene all in one container. The yeast hybrid assay can be a yeast two hybrid assay or a yeast three hybrid assay. The activity of these genes can be measured by measuring the activity of their gene products. MEL1 encodes α-galactosidase, and the activity of this protein can be measured through many techniques known in the art (Buckholz and Adams, Mol. Gen. Genet. 182: 77-81, 1981; Lazo, et al., Eur. J. Biochem. 77: 375-82, 1977). Its presence can also be measured through the use of antibodies specific for this protein. LacZ encodes β-galactosidase, and the activity of this protein can also be measured through many techniques known in the art (Ryan, et al., Mol. Cell. Biol. 18: 1774-82, 1998; Melcher, et al., Gene 247: 53-61, 2000). As with α-galactosidase, its presence can also be detected through the use of antibodies specific for β-galactosidase. URA3 encodes orotidine-5′-phosphate decarboxylase, which allows for the survival of yeast cells in media lacking uracil. The activity of this gene can be measured through the viability of the yeast cells themselves.

The single container employed in the methods of the invention can be a well in a multiwell plate or a microtiter plate. The multiwell or microtiter plate can have 24, 96, 384, or 1536 wells. More specifically, could have as few as one, or as many as 10,000 wells. The yeast can be cultured and assayed in liquid media, for easier manipulation in a high throughput system using liquid handling robotics. Additionally, the assays can be performed on any surface other than a solid or a gel. In one embodiment, the assays can not be performed on a surface, wherein that surface is a solid or a gel.

In one preferred embodiment of the invention, the diploid yeast colony to be screened in a liquid media is first separated into aliquots in wells of a multiwell plate. First, the buffers and salts appropriate for the colorimetric measurement of either MEL1 or lacZ are added, and the color change is detected by measuring the appropriate absorbance wavelength. Subsequently, the buffers and salts appropriate for the other assay is added, and the second color change detected by measuring another absorbance wavelength. Next, a third reporter can be used that, when active, leads to the survival of the cells in restrictive media. For example, URA3 could be used with media lacking uracil. Other reporters that affect the survival of yeast cells in certain restrictive media could also be used. The viability of the cells can be measured while measuring the result for either or both calorimetric assays. Those skilled in the art will recognize that the order of detection assays employed in the methods of the invention can be varied without impairing performance.

Yeast Two Hybrid Assay

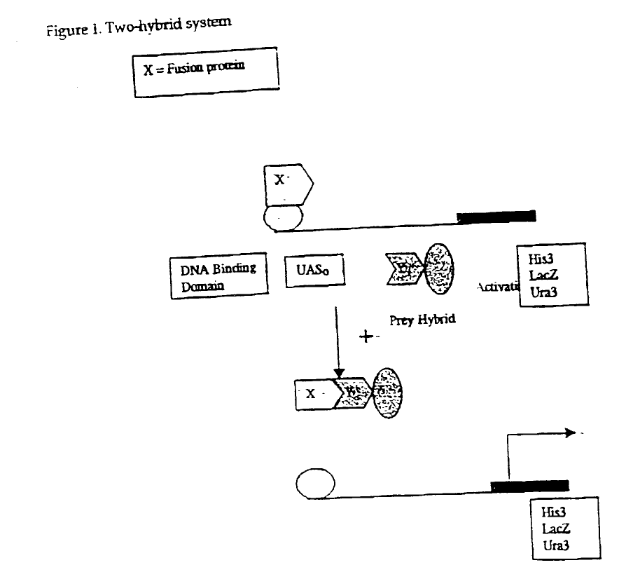

In one aspect, the invention is generally drawn to a yeast two hybrid (Y2H) screening assay format (the “sequential triple-reporter assay”) in which the readouts from the URA3, MEL1 and lacZ reporter genes are measured sequentially or simultaneously in a single microtiter plate. The yeast two hybrid assay is summarized generally in FIG. 1. The invention maximizes the amount and quality of data generated by high-throughput screening.

In one embodiment, the assay is used for automated, high-throughput screening for protein-protein interactions. The assay can use the Gal4-based Y2H system and is designed for the analysis of small culture volumes (55 μl) in 384-well plates (thus, making the assay inexpensive, since the estimated cost per assay is approximately 3.0 cents). The assay also requires only two liquid handling steps and can be used for the comprehensive mapping of protein-protein interactions occurring in organisms with large genomes.

In another embodiment, the assay is used to validate putative “interactor” colonies. In various embodiments, the interaction can be detected initially on selective agar medium or directly in combination with the interaction-mating approach.

In another embodiment of the invention, the haploid Y2H host strains contain both the MEL1 and lacZ reporter genes. Sequential α-/β-galactosidase assays can be employed further to test for self-activating bait and prey plasmids directly in their respective haploid host strains prior to mating. The assay according to the invention, yields data from three different reporter genes thereby reducing false positive results.

In some embodiments, the Y2H assay of the invention is performed in a liquid medium. Specifically, diploid colonies can be inoculated into liquid medium, and incubated there. Some time later the cells can be resuspended and an aliquot mixed with glycerol for measurement of the activity of the reporter genes. The liquid medium can be contained in a well within a multiwelled plate. The aliquot can also be transferred to a well of another multiwelled or microtiter plate. In some other embodiments, the multiwelled or microtiter plate can include as many as 384 wells, and the glycerol can be mixed with the aliquot at 50% v/v. In another embodiment, the initial culturing in liquid medium can be performed in as little as 100 μl. Alternatively, the aliquot taken can have a volume of about 45 μl.

The aliquot from the liquid medium can be measured to ascertain the activity of the reporter genes inside the diploid colonies inoculated into the liquid medium, as described above. The absorbance of the aliquot at 660 nm is taken to quantify cell growth. α-galactosidase activity encoded by MEL1 is measured by adding a substance that changes color with increased activity of α-galactosidase, i.e., p-nitrophenyl α-D-galactopyranoside, along with the appropriate salts and buffers. The color change of p-nitrophenyl α-D-galactopyranoside can be measured by observing the change in absorbance at 410 nm. Those skilled in the art will recognize that more than one measurement of absorbance can be made simultaneously. The β-galactosidase activity of the lacZ reporter gene can also be measured by adding a substrate of β-galactosidase that changes color with increasing β-galactosidase activity, such as red-β-D-galactopyranoside, along with the appropriate salts and buffers. The color change of red-β-D-galactopyranoside can be measured by observing the change in absorbance at 578 nm.

In another embodiment of the invention, the interaction of a bait and prey protein leads to the activation of a triple-reporter system. The triple-reporter system may include the URA3, MEL1, and lacZ reporter genes. The URA3 reporter gene is detected by selective growth of diploid cells in liquid medium lacking uracil. This can be measured by measuring absorbance at 660 nm. Assays of MEL1 and lacZ activity are described above, and can be combined with this assay for selective growth. The activation of the triple-reporter system can be measured in any order or can be measured simultaneously.

In a preferred embodiment of the invention, the detection assay of the three reporter genes is performed in one container, wherein the container can be a well in a multiwell or a microtiter plate. The conditions for assaying the activity of the MEL1 and lacZ genes are very different. Those skilled in the art have not previously been able to perform these assays in the same container. The present invention includes a method used to perform both assays in the same container. Which makes the yeast two hybrid system more amenable to high throughput screening. The method allows the assays to be performed in liquid, and require only one container. These conditions make this method amenable to robotic manipulation.

In another aspect, bait and prey plasmids can also be introduced into haploid strains, which are then mated to form diploid strains with both bait and prey plasmids.

Yeast Three Hybrid Assay

The invention is also broadly drawn to a liquid yeast three hybrid (Y3H) reporter assay using the triple reporter system in one container. For example, the container can be a well in a 96-well microtiter plate. The triple reporter system can be used in conjunction with the Y3H assay to detect a small-ligand protein interaction. As described above, the URA3, MEL1 and lacZ reporters can be used and can be detected sequentially or simultaneously. Specific protocols for their detection are disclosed below.

The liquid assay permits detection of reporter-gene trans-activation in two days, which is significantly less time (3 days to 1 week) than that required for similar assays on agar medium. Furthermore, because the culture volume required for the liquid assay is small micromolar concentrations of drug can be achieved without using large absolute quantities of the chemical compound. This may be important if drug screening is performed using combinatorial libraries of compounds, where the amount of each unique drug is limited. Moreover the assay described is compatible with smaller culture volumes (as little as 50 μl) and cell growth in 384-well plates, which provides the opportunity for further economy with chemical compounds. Because the assay is performed using liquid cultures it eliminates tedious preparation of drug-containing agar medium and makes Y3H screening accessible to automated high-throughput screening using liquid-handling robots.

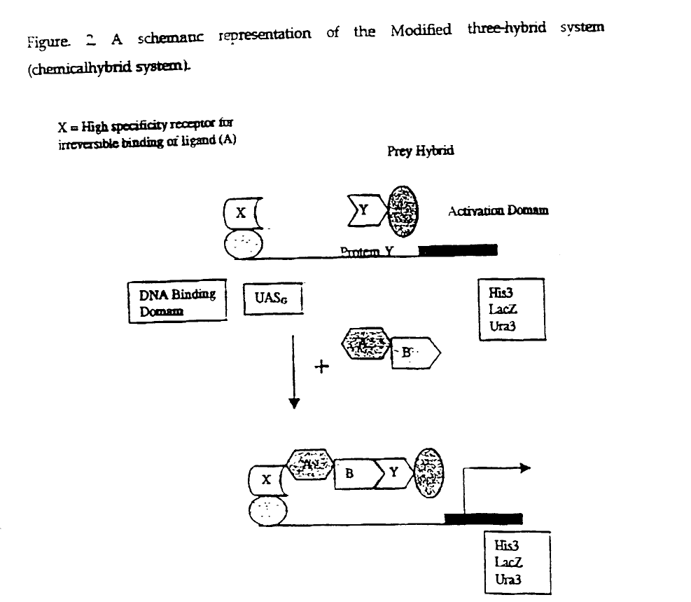

The yeast three hybrid screening system of the invention, in one embodiment, operates through the irreversible (covalent) interaction of a small molecule (ligand A in FIG. 2) with a fusion protein that contains a DNA binding domain, and a protein known to form an irreversible interaction with ligand A. Ligand A interacts with ligand B, as shown in FIG. 2. This interaction can be covalent, or non-covalent. In one preferred embodiment, the interaction is a high affinity interaction (i.e. covalent). In another preferred embodiment, the construct made up of the DNA binding/ligand A binding fusion protein, ligand A and ligand B is used to screen libraries of fusion proteins that are fused with a transactivation domain. When an interaction occurs between the substances fused to the DNA binding domain, and a protein in the library, the interaction can be measured through the activity of a reporter gene. In another embodiment of the invention, ligand A is not irreversibly bound to the fusion protein containing the DNA binding domain.

A significant difference exists between a three hybrid system in which ligand A is covalently bound to the DNA binding domain fusion protein, and one that is not covalently bound. When ligand A is covalently bound the DNA binding domain fusion protein, it can be used to detect interactions of lower affinity with ligand B, than can be detected if it is not covalently bound. A covalently bound ligand A localized ligand B to the DNA binding fusion protein constantly, while a reversibly bound ligand A only localizes ligand B this way when it is on the DNA binding domain fusion protein. This on/off ratio is dependent upon the affinity ligand A has for the DNA binding domain fusion protein. The higher the affinity the greater the proportion of the time ligand A is “on” the DNA binding domain fusion protein, and the lower the affinity the ligand B/activation domain fusion protein affinity can be and still have an interaction. If the bond between ligand A and the DNA binding domain fusion protein is covalent, then ligand A is essentially on the DNA binding domain fusion protein all of the time. This maximizes the lowest affinity that the interaction between ligand B and other proteins that can be detected.

In another preferred embodiment of the invention, the unknown component in the assay may be either the small molecule contained in the hybrid ligand, or one of the hybrid proteins (or both small molecule and protein). There is no requirement that the unknown component be purified prior to the screening assay. Indeed, it is expected that the unknown component be contained in a mixture containing a large number of components, some or all being unidentified. These interactions may be determined in vivo or in vitro when the chemical hybrid complex triggers the expression of at least one reporter gene that can be detected by an appropriate assay.

These methods of the invention can be used for: (1) determining the identity of target molecules having a binding affinity with a known small molecule where the small molecule has pharmacologic activity and where the target molecules may be suited for therapeutic intervention in a variety of disease states; (2) determining the identity of a small molecule capable of direct binding to a known target molecule where the identified small molecules may be suitable as therapeutic agents; (3) determining the identity of a small molecule capable of binding competitively to a known target molecule in the presence of a hybrid molecule so as to inhibit the binding between the target and the preselected small molecule; (4) developing a high throughput pharmacological assay in a number of cell types and organisms to screen for drug candidates; and (5) selecting novel small molecule for binding novel targets with high affinity using an iterative process of direct and competitive screening steps. For example, a known small molecule may be used to identify a target and subsequently the target may be used to identify a novel small molecule. This approach can provide novel small molecule pharmacologic agents and may also provide highly specific reagents for use in screening for small molecules in the environment.

The method identified here as the chemical-hybrid system includes the step of providing a hybrid molecule consisting of two ligands identified as ligand A and ligand B that are linked together, wherein ligand A has a specificity for a first predetermined target and can form an irreversible (covalent) bond; and ligand B is the small molecule (FIG. 2). In another preferred embodiment of the invention, a set of novel hybrid molecules which form an irreversible bond between the ligand A/ligand B molecule and the predetermined target resulting in changing the three hybrid system to a two-hybrid type of system (referred as chemical-hybrid system here). There are several obvious advantages of the new screening system such as enhanced sensitivity, specificity and thus allowing screening of wide range of ligands and proteins (permit detection of both strong and weak ligand-protein interactions).

### EXAMPLES

### Example 1

### Sequential Triple Reporter Assay: A “One Plate/Three-Reporter” Assay Format for Detecting and Validating Yeast Two-Hybrid Interactions

A. General Methods

Growth medium. Yeast cells were grown in liquid YPD (4001-016) or selective DOB medium (4025-012) with supplements (4530-912) (Qbiogene, Carlsbad, Calif.) or on rich YPD or selective CM agar plates (Teknova, Halfmoon Bay, Calif.). Mating reactions were performed in liquid medium containing 2X YPD as described below.

Yeast strains and plasmids. The haploid Y2H host strains YULH (MATα) and N106r (MATa) have been described (Uetz, et al., Nature 403: 623-7, 2000); both strains contain the MEL1 and GAL1::lacZ reporters while YULH alone contains the GAL1::URA3 reporter. Haploid strains, containing fusions of Gal4_{BD} or Gal_{AD} to full-length Drosophila genes were mated to produce CGY diploid control strains. Bait genes were fused to GAL4_{BD} in vector pDBGal4CAM (Stratagene, La Jolla, Calif.) while preys were fused to GAL4_{AD} in vector pACT2 (Clontech, Paulo Alto, Calif.). The hybrid proteins encoded in diploid strains CGY-1D and CGY-3D participate in a strong and weak interaction, respectively, while those encoded in diploid strains CGY-2D and CGY23D participate in a medium-strength interaction. The hybrid proteins encoded in strain CGY-4D do not interact, while a chromosomal mutation in this strain confers uracil prototrophy independent of a Y2H interaction.

Sequential triple-reporter assay. Diploid colonies (of strains CGY-1D to -4D) were inoculated into selective liquid medium (lacking tryptophan, leucine and uracil; 100 μl) in a flat-bottom 384-well microtiter plate (Bio-one GH1162/FE6191; Greiner Lake Mary, Fla.) and incubated at 30° C. for 3 days in a sealed container. The cells were resuspended and an aliquot of each culture (45 μl) was transferred to a second 384-well plate containing glycerol (50% v/v), mixed and archived at −80° C. As an option at this stage, the A₆₆₀ of the cultures remaining in the original (assay) plate were measured to quantify cell growth (see alternative procedure below). To measure α-galactosidase activity encoded by MEL1, 20 μl of Buffer ZLX (3.75X Z-buffer [1X: 16.1 g/l Na₂HPO₄.7H₂O, 5.5 g/l NaH₂PO₄.H₂O, KCl 0.75 g/l, MgSO₄.7H₂O, 0.246 g/l, pH 7.0], 23.4 mM X-a-Gal [p-nitrophenyl α-D-galactopyranoside, N0877, Sigma], 188 U/ml lyticase [62982; Fluka, Buchs, Switzerland] was added to the cultures, mixed and incubated at room temperature for 60-180 min. Color-development (at A₄₁₀ or ΔA₄₁₀/A₆₆₀) was measured using a PowerWaveₓ Select platereader with KC4 software (BioTek Instruments Winooski, Vt.). The ΔA₄₁₀/A₆₆₀ reading provides a measurement of β-galactosidase activity correlated to the amount of biomass present and eliminates the necessity for an independent A₆₆₀ measurement to correct for growth (see above). To subsequently measure β-galactosidase activity encoded by the lacZ reporter gene, 25 μl of Buffer CPRG-N (0.5 mg/ml CPRG [chlorophenol red-β-D-galactopyranoside, 884 308, Roche Molecular Biochemicals, Indianapolis, Ind.], 0.8% IGPAL CA-630; Sigma) was added to each well, mixed and incubated at room temperature for 60 min (or longer, up to an overnight incubation) prior to measurement of color-development (A₅₇₈ or ΔA₅₇₈/A₆₆₀). Data was processed in Microsoft Excel by subtracting the background signal generated by negative control strain CGY-4D from the signals obtained from positive interactor strains.

Interaction-Mating. Bait and prey plasmids were recovered from the diploid control strains CGY-1D, -23D, -3D and -4D (RPM yeast plasmid isolation kit 2069-000, QBiogene) and reintroduced into strain YULH and N106r, respectively by standard procedures (Schiestl and Gietz, Curr. Genet. 16: 339-46, 1989). Diploid cells of strain CGY-1D, -23D, -3D and -4D were regenerated by interaction mating as described (Buckholz, et al., J. Molec. Microbiol. Biotech. 1: 135-40, 1999) with the following modifications. The relevant haploid transformant cells were grown in selective medium (3 ml) by incubation at 30° C. overnight with agitation. Crosses were made by mixing 2 μl of a saturated MATa (bait) culture with 8 μl of the appropriate MATα (prey) culture in 10 μl of medium containing 2X YPD in a V-bottomed 96-well plate (FE1161; DOT Scientific Inc.) followed by incubation at 30° C. overnight. The mating reactions were resuspended, diluted 100-fold in selective medium (lacking tryptophan, leucine and uracil), inoculated into a flat-bottom 384-well plate (100 μl culture volume) and incubated at 30° C. for 3 days in a sealed container to permit selective outgrowth of diploid cells. (Dilution of rich medium present in the mating reaction is necessary to prevent non-selective outgrowth of non-interactor cells (Buckholz, et al., J. Molec. Microbiol. Biotech. 1: 135-40, 1999)). The diploid strains CGY-1D, -23D, -3D and -4D were cultured in an identical manner to that described above except that 10 μl of the saturated diploid culture was added to the 2X YPD-containing medium. Cells were archived and analyzed by sequential α- and β-galactosidase assays as described above.

B. Validation of Y2H Interactions Using a Sequential Triple-Reporter Assay.

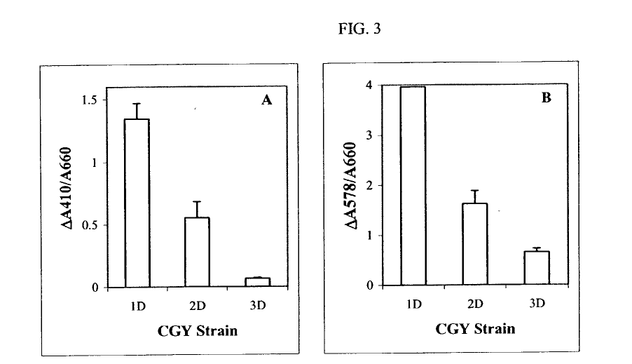

Blue-colored diploid colonies containing putative Y2H interactions were selected on agar medium lacking uracil and containing X-gal as described by Uetz, et al. (Nature 403: 623-27, 2000). To validate and quantify the strength of protein-protein interactions occurring in these blue colonies, a liquid assay was devised to permit readout from three different reporter genes conveniently in a single 384-well microtiter plate. Activation of the URA3 reporter gene was first confirmed by selective growth of diploid cells in liquid medium lacking uracil. Subsequently, α-galactosidase activity expressed from the MEL1 reporter was measured in the presence of Z-buffer (pH 7.0) and lyticase (FIG. 3A). Under these conditions development-development (colorless to yellow) occurs in concert with the hydrolysis of X-a-Gal (without the requirement for subsequent pH adjustment) while the concomitant cell lysis increases signal-strength via release of intracellular enzyme. Following measurement of development-development, detergent and CPRG was added to each well to promote completion of cell lysis and rapid detection of β-galactosidase activity (FIG. 3B). The rate of development in each assay could be adjusted conveniently by varying the concentration of substrate and/or lyticase, while the signal ratio between strong, medium-strength and weak interactor strains was generally similar in the α- and β-galactosidase assays.

C. Y2H-Screening by Combined Interaction Mating and Sequential Triple-Reporter Assay.

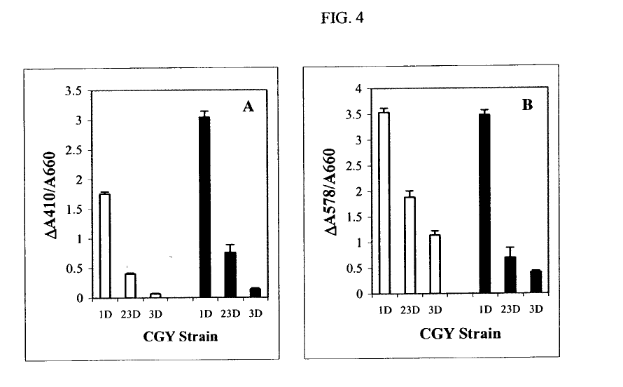

The sequential α-/β-galactosidase assay format was also used for the primary detection of protein-protein interactions by Y2H matrix (1×1) screening. Interaction-mating of MATa and MATα haploid cells, containing bait and prey plasmids respectively, was performed in V-bottom plates. Diploid cells were inoculated into 100 μl of selective medium following a 100-fold dilution of the mating mix. A subsequent period of selective outgrowth was included to test for the activation of the URA3 reporter gene and to enrich for diploid cells. Using this approach the strength of the protein-protein interactions detected in newly generated diploids correlated well with the strength of those detected in the corresponding diploid cells grown directly without mating (FIGS. 4A and 4B). Thus this assay format provides reliable quantitation of protein-protein interactions following a 1×1 interaction mating assay.

### Example 2

### A Y3H Liquid Reporter Assay: A Microtiter Plate-based Yeast Three Hybrid Reporter Assay for the Detection of Protein-Ligand Interactions in Liquid Yeast Cultures

A. General Methods

Growth medium. Yeast cells were grown in liquid SC-trp-leu-ura medium, which is DOB medium (4025-012) supplemented with CSM-leu-trp-ura (4530-912) (Qbiogene, Carlsbad, Calif.). Where required, uracil (Sigma U0750) was added to SC-trp-leu-ura medium to a final concentration of 20 μg/ml to generate SC-trp-leu. SC-leu, SC-trp and SC-trp-leu agar plates were obtained from Teknova (Halfmoon Bay, Calif.; #C3040, 3060 and 3220, respectively).

Yeast strains and plasmids. The haploid Y2H host strains YULH (MATa) and N106r (MATa) have been described (Uetz, et al., Nature 403: 623-27, 2000); both strains contain the GAL1::lacZ reporter gene and are mutated for the chromosomal URA3 gene resulting in uracil auxotrophy, while YULH alone contains the GAL1::URA3 reporter. Strain CGY-201-202D was constructed as follows. Plasmid pPAS201-1 was first constructed by fusing the F620S C656G double mutant rat glucocorticoid receptor hormone binding domain (rGR; amino acids 524-795; Licitra and Liu, Proc. Natl. Acad. Sci. USA 93: 12817-21, 1996) to Gal4DB in vector pGBT9 (Clontech) and was introduced into strain YULH. Plasmid pPAS202-1 was constructed by fusing FKBP12 (Licitra and Liu, Proc. Natl. Acad. Sci. USA 93: 12817-21, 1996) to Gal4AD in vector pGAD-GH (Clontech) and was introduced into strain N106r. Plasmids were introduced into yeast by standard methods. Strain CGY-201-202D expressing both rGR and FKBP12 was generated by mating a transformant strain of YULH containing pPAS201-1 with a N106r transformant containing pPAS202-1, and selecting for diploid cells containing both plasmids on SC-leu-trp agar. The control strain CGY-201-GHD expressing rGR but lacking FKBP12 was generated by mating YULH containing pPAS201-1 with N106r containing the empty pGAD-GH vector and selecting for diploid cells.

Y3H liquid reporter assay. A stock of 1 M dexamethasone-FK506 (dex-FK506; Oxford Asymmetry) in 100% DMSO was diluted to generate a final 3-fold dilution series in 5% DMSO (1000 μM, 333 μM, 111 μM, 37 μM and 12 μM). Duplicate aliquots (15 μl) of each drug dilution and a drug-free control (5% DMSO) were distributed to a transparent, 96-well flat-bottomed microtiter plate (Greiner Lake Mary, Fla.). A fresh saturated culture of strain CGY201-202D (grown at 30° C. overnight in SC-leu-trp) was diluted 10-fold in SC-leu-trp-ura and aliquots (135 μl of culture) were added to each drug sample in the microtiter plate. A similar procedure was performed for strain CGY-201-GHD and the cultures were incubated at 30° C. in the microtiter plate without agitation. Following 2 days of incubation, the cells were resuspended by aspiration and the amount of cell growth in the absence of added uracil (supported by transactivation of the GALUAS-URA3 reporter gene) was determined by measuring the A660 of the cultures using a PowerWavex Select platereader with KC4 software (BioTek Instruments Winooski, Vt.). To measure β-galactosidase activity resulting from trans-activation of the GALUAS-lacZ reporter, an equal volume of CPRG assay buffer was added to each culture. CPRG assay buffer is 2X Z-buffer (1X; 16.1 g/l Na₂HPO₄.7H₂O, 5.5 g/l NaH₂PO₄.H₂O, KCl 0.75 g/l, MgSO₄.7H₂O, 0.246 g/l, pH 7.0) containing CPRG (chlorophenol red-β-D-galactopyranoside; #884 308, Roche Molecular Biochemicals, Indianapolis, Ind.) at 2.0 mg/ml, IGPAL CA-630 (Sigma) at 0.4% and lyticase [62982; Fluka, Buchs, Switzerland] at 50 U/ml. Alternatively, an aliquot (50 μl) was removed from each culture in the 96-well plate and transferred to a transparent, flat-bottomed 384-well plate followed by the addition of an equal volume of CPRG assay buffer. Following incubation at room temperature β-galactosidase activity was analyzed by measuring development-development at Δ578/A660. The Δ578/A660 reading provides a measurement of β-galactosidase activity correlated to the amount of biomass present and eliminates the necessity for an independent A660 measurement to correct for growth. Data (A660 and Δ578/A660 readings) were processed in Microsoft Excel by subtracting the background signal generated by the negative control strain CGY-201-GHD at each drug concentration, from the corresponding signals generated by the test strain, CGY-201-202D.

B. Validation of the Microtiter-Plate-Based Liquid Reporter Assay for the Y3H System.

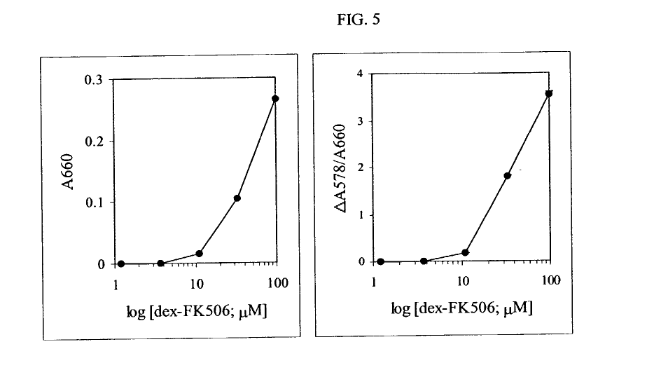

To demonstrate the veracity of a microtiter plate-based liquid reporter assay for the Y3H system, the bi-ligand dependent interaction of mutant rGR with FKBP12 was measured using yeast cells inoculated into liquid medium containing the hybrid molecule dexamethasone-FK506 (dex-FK506). All test cultures were incubated in 96-well plates and uracil was omitted from the (SC-leu-trp-ura) medium to test for trans-activation of the GALUAS-URA3 reporter gene present in the host cells. Cells expressing rGR but not FKBP12 were used as a negative control for reporter activation. Following incubation at 30° C. for 2 days, drug-induced cell growth mediated by trans-activation of the GALUAS-URA3 reporter was analyzed by measuring culture absorbance at 660 nm. Consistent with a bi-ligand-induced interaction between rGR and FKBP12, trans-activation of GALUAS-URA3 occurred in a dex-FK506 dose-dependent manner Similar results were obtained if an equal volume of CPRG assay buffer was added to cultures directly in the 96-well growth plate. Because the host yeast cells contained a second reporter gene, GALUAS-lacZ, the drug-induced expression of β-galactosidase activity was tested by measuring the hydrolysis of the chromogenic substrate, CPRG, following cell lysis directly in the microtiter plate. As for GALUAS-URA3, the GALUAS-lacZ reporter was trans-activated in a dose-dependent manner by dex-FK506 (FIG. 5B). For both the GALUAS-URA3 and GALUAS-lacZ, reporter-gene the trans-activation was detected at a concentration of dex-FK506 of less than 10 μM though signal-strength increased significantly as dex-FK506 concentration increased thereafter up to a 100 μM (FIGS. 5A and 5B). Similar results were obtained for the GALUAS-lacZ reporter gene when cells were inoculated into liquid (SC-leu-trp) medium containing uracil.

C. Identification of a Small Molecule Capable of Binding to a Selected Target Molecule

A population of yeast cells which have previously been transformed with vectors as described above where the first hybrid protein any of the target receptor (e.g., cyclooxygenase, transpeptidase) fused to LexA DNA-binding domain, and the second hybrid protein is rat glucocorticoid receptor (or FKBP12) fused to a transcriptional activator module and the reporter gene is Lac Z (and Ura3). A 96-well plate is prepared such that each well contains a single member of the hybrid ligand library composed of ligand A covalently (e.g., aspirin, β-lactams, vigabatrin, and fluorescein) linked to a library of small molecules. The transformed yeast is grown in each well and a blue coloration is looked for (growth of colonies with Ura3). Those wells expressing the reporter gene are identified and structural information on the corresponding hybrid ligand is retrieved.

D. Competitive Assay for Identifying a Small Molecule Ligand having a Binding Affinity for a Known Target.

A population of yeast cells which have previously been transformed with vectors according to the methods described above in this Example, are placed in a 96 well dish. These yeast cells were transformed with DNA encoding a first hybrid protein which is the target receptor (e.g., cyclooxygenase, transpeptidase) fused to LexA DNA-binding domain, and a second hybrid protein which is glucocorticoid receptor (and FKBP12) fused to a transcriptional activator module and a third vector containing the reporter Lac Z gene (and Ura3). A single member of a ligand library covalently linked to a hybrid ligand is added to each well containing the yeast. Those wells which are identified as having a blue coloring are scored as negative while those wells that appear white were scored positive. Control wells having either hybrid ligand only or no hybrid molecule are included. The samples are identified according to the absence of expression of the reporter gene; and the ligand from the library is characterized so as to determine its structure information.

E. Assay for Identifying a Diagnostic Reagent for Screening for Small Molecule Contaminants in the Environment.

A cDNA transcriptional activator fusion library is prepared from immune cells (B-cells) capable of producing antibodies to a specific small molecule contaminant, in this case, DDT. Using the screening assay described in this Example, a hybrid molecule is formed from irreversible ligand A/DDT. Yeast cells are transformed accordingly with the cDNA fusion library, a vector encoding the hybrid protein containing binding domain of the target receptor and a vector encoding the reporter gene Lac Z (and Ura3) and the hybrid ligand is introduced so as to identify target molecules. The positive clones are identified by the blue coloration (and growth of colonies). The vector containing the cDNA from positively staining cells is isolated and the protein product utilized as a reagent in environmental screening assays to detect DDT with high affinity.

F. A Chemical Hybrid Screening Assay Kit

A kit is prepared that contains a plasmid encoding the LexA DNA binding module fused to the target receptor according to the methods described in this Example; a plasmid encoding the transcriptional activation domain fused to fragments in a cDNA library: and a reporter plasmid containing Lac Z, Ura3, GFP or luciferase. The cDNA library for use in the kit is selected from a variety of sources including T-cells, cardiac cells and liver cells. the choice being dependent on the characteristics of the potential target protein and the small molecule. The kit contains a conserved ligand for reacting with a small molecule to form a hybrid molecule by standard coupling procedures described above. Although a number of linkages may be exploited including ester, ether and amide bonds. In addition, the kit provides an environment. in this case, yeast cells, for permitting the chemical hybrid screening assay to occur.

### OTHER EMBODIMENTS

The present invention is not to be limited in scope by the specific embodiments described herein. Indeed, various modifications of the invention in addition to those described herein will become apparent to those skilled in the art from the foregoing description an accompanying figures. Such modifications are intended to fall within the scope of the appended claims. Various publications are cited herein, the disclosures of which are incorporated by reference in their entireties.

We claim:

1. A method of performing a yeast hybrid assay, the method comprising detecting the activation of at least three reporter genes using detection assays specific for each reporter gene, wherein the at least three detection assays used to detect the different reporter genes are performed in the same container.

2. The method of claim 1, wherein the yeast hybrid assay is selected from the group consisting of a yeast two hybrid assay and a yeast three hybrid assay.

3. The method of claim 1, wherein the reporter genes are selected from the group consisting of URA3, MEL1, and lacZ.

4. The method of claim 1, wherein the detection assay used to detect the reporter genes measures the activity of a protein product of the reporter gene.

5. The method of claim 4, wherein the protein product is selected from the group consisting of orotidine-5′-phosphate decarboxylase, α-galactosidase, and β-galactosidase.

6. The method of claim 1, wherein the detection assay used to detect the reporter genes detects the presence of a protein product of the reporter gene.

7. The method of claim 6, wherein the protein product is selected from the group consisting of orotidine-5′-phosphate decarboxylase, α-galactosidase, and β-galactosidase.

8. The method of claim 1, wherein the assays are not performed on a surface.

9. The method of claim 8, wherein the surface is a gel or a solid.

10. The method of claim 1, wherein the assays are performed in a liquid medium.

11. The method of claim 1, wherein the container is selected from the group consisting of a well and a microtiter plate.

12. The method of claim 11, wherein the container is selected from the group consisting of a 24 well microtiter plate, 96 well microtiter plate, a 384 well microtiter plate, and a 1536 well microtiter plate.

13. A method of performing a yeast two hybrid assay, the method comprising detecting the interaction of a prey fusion protein and a bait fusion protein by the activation of at least three reporter genes each detected by specific detection assay, wherein at least three or more detection assays are performed in one container.

14. The method of claim 13, wherein the reporter genes are selected from the group consisting of URA3, MEL1, and lacZ.

15. The method of claim 13, wherein the detection assay used to detect the reporter genes measures the activity of a protein product of the reporter gene.

16. The method of claim 15, wherein the protein product is selected from the group consisting of orotidine-5′-phosphate decarboxylase, α-galactosidase, and β-galactosidase.

17. The method of claim 13, wherein the detection assay used to detect the reporter genes assays the presence of the protein product of the reporter gene.

18. The method of claim 17, wherein the protein product is selected from the group consisting of orotidine-5′-phosphate decarboxylase, α-galactosidase, and β-galactosidase.

19. The method of claim 13, wherein the assays are performed in a liquid medium.

20. The method of claim 13, wherein the assays are not performed on a surface.

21. The method of claim 20, wherein the surface is a gel or a solid.

22. The method of claim 13, wherein the container is selected from the group consisting of a well and a microtiter plate.

23. The method of claim 22, wherein the container is selected from the group consisting of a 24 well microtiter plate, 96 well microtiter plate, a 384 well microtiter plate, and a 1536 well microtiter plate.

24. A method of performing a yeast three hybrid assay, the method comprising detecting the interaction of a bait fusion protein, a ligand, and a prey fusion protein by the activation of at least three reporter genes each detected by specific detection assay, wherein at least three or detection assays are performed in one container.

25. The method of claim 24, wherein the reporter genes are selected from the group consisting of URA3, MEL1, and lacZ.

26. The method of claim 24, wherein the detection assay used to detect the reporter genes measures the activity of a protein product of the reporter gene.

27. The method of claim 26, wherein the protein product is selected from the group consisting of orotidine-5′-phosphate decarboxylase, α-galactosidase, and β-galactosidase.

28. The method of claim 24, wherein the detection assay used to detect the reporter genes detects the presence of a protein product of the reporter gene.

29. The method of claim 28, wherein the protein product is selected from the group consisting of orotidine-5′-phosphate decarboxylase, α-galactosidase, and β-galactosidase.

30. The method of claim 24, wherein the assays are not performed on a surface.

31. The method of claim 30, wherein the surface is a gel or a solid.

32. The method of claim 24, wherein the assays are performed in a liquid medium.

33. The method of claim 24, wherein the container is selected from the group consisting of a well and a microtiter plate.

34. The method of claim 33, wherein the container is selected from the group consisting of a 24 well microtiter plate, 96 well microtiter plate a 384 well microtiter plate, and a 1536 well microtiter plate.
